(19) United States
(12) Patent Application Publication
(10) Pub. No.: US 2013/0102056 A1
(43) Pub. Date: Apr. 25, 2013

(54) LIQUID CURTAIN PHOTOBIOREACTORS

(71) Applicant: EXXONMOBIL RESEARCH AND ENGINEERING COMPANY, Annandale, VA (US)

(72) Inventors: RONALD SURYO, Fairfax, VA (US); JOHN T. WYATT, Chester, CT (US)

(73) Assignee: EXXONMOBIL RESEARCH AND ENGINEERING COMPANY, Annandale, NJ (US)

(21) Appl. No.: 13/629,966

(22) Filed: Sep. 28, 2012

Related U.S. Application Data
(60) Provisional application No. 61/542,336, filed on Oct. 3, 2011.

(51) Int. Cl. C12M 1/00

(52) U.S. Cl. CPC C12M 21/02
USPC 435/252.1; 435/292.1; 435/257.1

(57) ABSTRACT

A relatively low cost, highly productive, scalable culture vessel, and method to grow photosynthetic organisms, in particular autotrophic algae, is provided. The culture vessel can be constructed of a transparent plastic enclosure. One or more thin sheets, films, droplets, or streams of a free falling suspension of organism in liquid media can be continuously formed within an enclosure. The free falling media can fall into a collector or pool at the bottom of the enclosure. The media can then be recirculated to the top of the enclosure to once again form into a free falling sheet, curtain, droplets, etc. The organisms (e.g., algae) can then be harvested from a collector.

### CROSS-REFERENCE TO RELATED APPLICATIONS

This application claims the benefit of provisional U.S. application Ser. No. 61/542,336, filed Oct. 3, 2011, the entirety of which is hereby incorporated by reference.

### FIELD OF THE INVENTION

The present invention relates to culture vessels for growing algae, More particularly, the invention relates to culture vessels to grow algae at high density.

### BACKGROUND OF THE INVENTION

Microalgae and cyanobacteria (hereinafter “algae”) primarily require simple mineral nutrients and carbon dioxide (CO₂) for growth and reproduction. With over 40,000 identified species, microalgae represent a very diverse group of organisms. Through photosynthesis, microalgae convert water and CO₂ into bioproducts. Examples of microalgae bioproducts include biofuels, pigments, proteins, fatty acids, and carbohydrates to name a few. Algae exhibit a growth potential an order of magnitude greater than higher plants because of their extraordinarily efficient light and nutrient utilization.

Although application of algae to produce renewable biofuels (in both liquid and gaseous form) and other high-value products is scientifically and environmentally sound, the economic viability of such algal applications is determined largely by the efficiency and cost-effectiveness of industrial-scale culture vessels in which algae grow and proliferate. Although hybrid systems are known, these growth vessels, or photobioreactors, are typically categorized into two types referred to as 1) open systems (most often in the form of raceway ponds although other types of ponds, such as circular ponds, have been employed) and 2) closed systems (alternatively referred to herein as “closed photobioreactors” or “closed PBRs”).

Raceway ponds are typically shallow ponds, about 30 cm deep, constructed as a continuous loop around which water containing algae is circulated mechanically via a paddle wheel. These and other open systems have the advantage of being relatively simple and cheap in construction and maintenance. Open systems, however, have disadvantages associated with their use that limit the productivity of algal growth. First, open ponds do not utilize light efficiently. Algae absorb light and the depth of water required for effective circulation is generally deeper than incident light can penetrate given the degree of algae absorbance. Second, mixing in open ponds dissipates rapidly as algae culture moves away from the paddlewheel and essentially becomes plug flow throughout the majority of the pond. Third, open ponds are subject to environmental factors such as wind, rain, temperature and evaporation. Fourth, open ponds are prone to contamination by airborne micro-organisms and dust that tower productivity even further and sometimes cause culture failure. The poor light utilization, poor mixing, limited environmental control and contamination issues result in relatively tow biomass and bioproduct productivity. Typical biomass productivity for an open pond is on the order of 10-20 g/m²/day of dry microalgae. These significant productivity drawbacks prompted the development of closed photobioreactors.

Closed photobioreactors are typically constructed of transparent tubes or containers in which the algae culture is mixed by either a pump or air/gas injection. A number of closed photobioreactors have been proposed and developed.

One type of closed photobioreactor is a tubular photobioreactor. Tubular photobioreactors generally comprise serpentine or helical tubing made of glass or plastic, coupled to a gas exchange vessel (wherein carbon dioxide (CO₂) and/or air are typically added and oxygen (O₂) is typically removed). The gas exchange vessel is typically connected to the ends of the tubing. Recirculation of the algae culture between the gas exchange vessel and the tubing is generally performed by a pump or an air-lift. In tubular photobioreactors, incident light is generally able to enter the tubing from multiple directions. Further, the tubing diameter is typically thinner than the depth of an open pond. Therefore, incident tight is utilized more effectively. In addition, mixing and environmental control is easier to accomplish in tubular photobioreactors. Because of their improvement in light path, mixing, and environmental control, tubular photobioreactors not only considerably increase algal biomass productivity and optimal growth density, but also enable more algal species of commercial interest to grow and proliferate under more controllable conditions.

Tubular-type photobioreactors, however, also have a number of disadvantages. First, tubular photobioreactors tend to have a significant “dark zone or dark volume” associated with a degas reservoir/tank where the exchange of excess amounts of dissolved oxygen with carbon dioxide occur. Algal cells entering the dark zone cannot perform photosynthesis, but consume, through cellular respiration, cell mass previously assimilated under light. Second, tubular photobioreactors have the potential to accumulate, in the culture suspension, high concentrations of molecular oxygen evolved from photosynthesis—because there is nowhere for oxygen to go until it reaches the gas exchange vessel. This, in turn, inhibits photosynthesis and thus biomass production potential. Third, mechanical pumps that are commonly employed by tubular photobioreactors to facilitate culture mixing and circulation within the long tubes are, by necessity, powerful and can cause cell damage. Due to the hydrodynamic stress created by the mechanical pumps, only a limited number of algal species are able to thrive in a tubular bioreactor. Fourth, tubular photobioreactors are expensive to operate. Fifth, tubular photobioreactors are still not sufficiently productive. Sixth, tubes in tubular photobioreactors have a high risk of biofouling—whereby algae and other materials accumulate on the interior surfaces of the tubes and reduce their transparency. Seventh, and finally, tubular photobioreactors are not readily scalable.

Another type of closed photobioreactor is a flat panel or flat plate-type. Flat panel photobioreactors are typically made of flat, rigid, smooth plastics. Flat panel PBRs yield higher algae productivity and higher optimal algae growth compared to raceway ponds and tubular PBRs. It has been shown that increasing the mixing rate in flat panel PBRs enhances the productivity through a mechanism known as intermittent light effect or “tight/dark cycle”. In other words, when the algae concentration in the flat panel is high, the algae near the surface of the flat panel experience higher tight intensity than the algae that reside further from the surface of the flat panel. As the mixing rate is increased, the algae move in and out of the light exterior and dark interior of the flat panel chamber. Flat panels PBRs, while giving moderate to high productivity, still require moderate to high energy input and are expensive to build and operate. Further, like tubular PBRs, flat panel PBRs suffer from biofouling and a tack of scalability.

For the production of biofuels from autotrophic algae to become economically viable, low cost, high-productivity, industrially scalable open or closed PBRs and related methods are needed. As stated, conventional open and closed PBRs are either: (i) cheap but yield low productivity and low concentration; or (ii) expensive and yield low to high productivity and low to high algae concentration.

### SUMMARY OF THE INVENTION

A relatively low cost, highly productive culture vessel for growing photosynthetic organisms (in particular autotrophic algae) is provided. The apparatus can comprise a number of components. A first component can be a partially open or fully closed enclosure that is at least partially transparent or translucent (preferably transparent). A second component can be a header, distribution plate, or distribution tubing (collectively, “header”) positioned in the upper portion of the enclosure, A third component can be one or more (preferably multiple) apertures in the header. A fourth component can be a collector section (a.k.a. collector), positioned in the bottom portion of the enclosure. A fifth component can be a main chamber positioned between the header and collector. In operation, a photosynthetic organism in a liquid growth medium (preferably water or nutrient enriched water) can be circulated to the header, thereafter flowing through the apertures, and then descending as one or more (preferably multiple) streams, sheets, or droplets through the main chamber toward the collector. Preferably, the media can thereafter be re-circulated to the header (e.g., by an internal or external pump, air lift, or other conventional means). The process can preferably be repeated multiple times, e.g., until a desired growth density of the organism in the media is achieved. The culture vessel can preferably also include other components typical in a PBR, such as gas inlet(s), gas outlet(s), feed port(s), harvesting port(s), and, optionally, one or more heat management components.

Also provided is a relatively low cost, highly productive method for growing a photosynthetic organism (in particular autotrophic algae). The method can comprise a number of steps. A first step can be providing nutrients (e.g., CO₂, metals, N) to a media (preferably water or nutrient enriched water) containing a photosynthetic organism (preferably algae). A second step can be circulating the media to a header. A third step can be forming the media into one or more (preferably multiple) descending streams, sheets, or droplets. A fourth step can be capturing the descending media in a collector. A fifth step can be re-circulating the media from the collector to the header. Preferably, steps two through five can be repeated multiple times.

The apparatus and method described herein can provide one or a number of significant advantages compared to conventional growth systems and methods. First, the continuously flowing, free falling, thin streams, sheets, or droplets of media can provide interfaces for natural gas exchange (e.g., CO₂ enrichment to the algae suspension and O₂ stripping from the algae suspension) and/or can provide the same type of light dilution and/or short light lath normally associated with an array of flat panel PBRs. This, in turn, can generate markedly higher algae concentrations, which can provide cost savings during the growth process (because less liquid media may be needed) and/or downstream harvesting operations (because less liquid media may need to be separated and/or recycled). Second, the free falling algae suspension can experience relatively low shear, reducing the probability of cell breakage. Third, within the main chamber, there can be substantially no biofouling on the surface where sunlight impinges the algae suspension because that surface is the algae suspension. This reduced biofouling, in turn, can result in comparatively lower maintenance/cleaning requirements and associated costs. Fourth, the apparatus of the invention can be more easily and cheaply mass produced, installed, and scaled up compared to conventional growth systems.

### BRIEF DESCRIPTION OF THE DRAWINGS

The following figures are for illustrative purposes only and are not ended to limit the scope of the present invention in any way:

FIG. 1 is a perspective view of a culture vessel constructed in accordance with some embodiments of the present invention.

FIG. 2 is a side view of a culture vessel constructed in accordance with some embodiments of the present invention.

FIG. 3 is a top view of an embodiment of a header of a culture vessel of the present invention.

FIG. 4 is a top view of an alternate embodiment of a header of a culture vessel of the present invention.

FIG. 5 is a side view of an alternate embodiment of a header of a culture vessel of the present invention.

FIG. 6 is a perspective view of a culture vessel constructed in accordance with some embodiments of the present invention.

FIG. 7 is a perspective view of an alternate embodiment of a collector section of a culture vessel of the present invention.

FIG. 8 is a side view of a turbine in an alternate embodiment of a collector section of a culture vessel of the present invention.

FIG. 9A-D show AFDW measurements versus time for Examples 1-4, respectively.

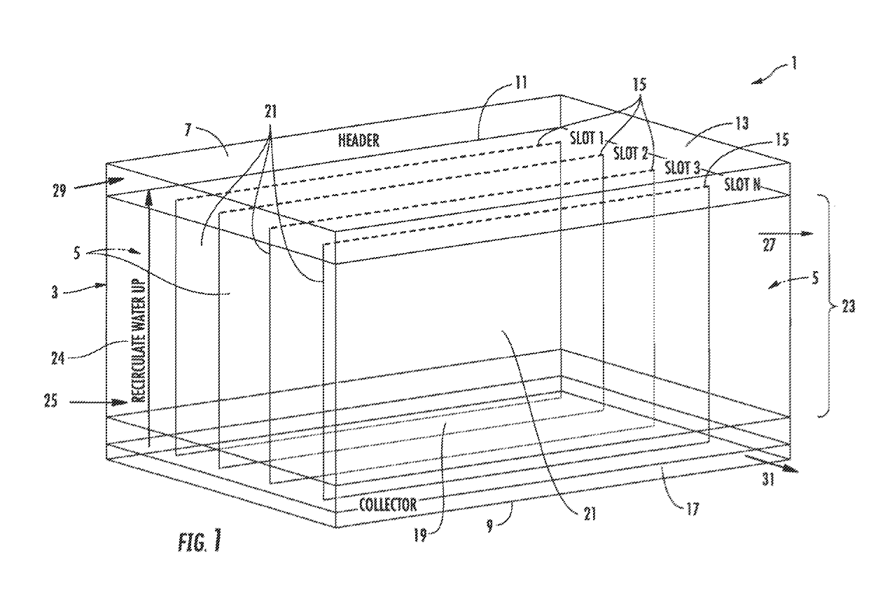

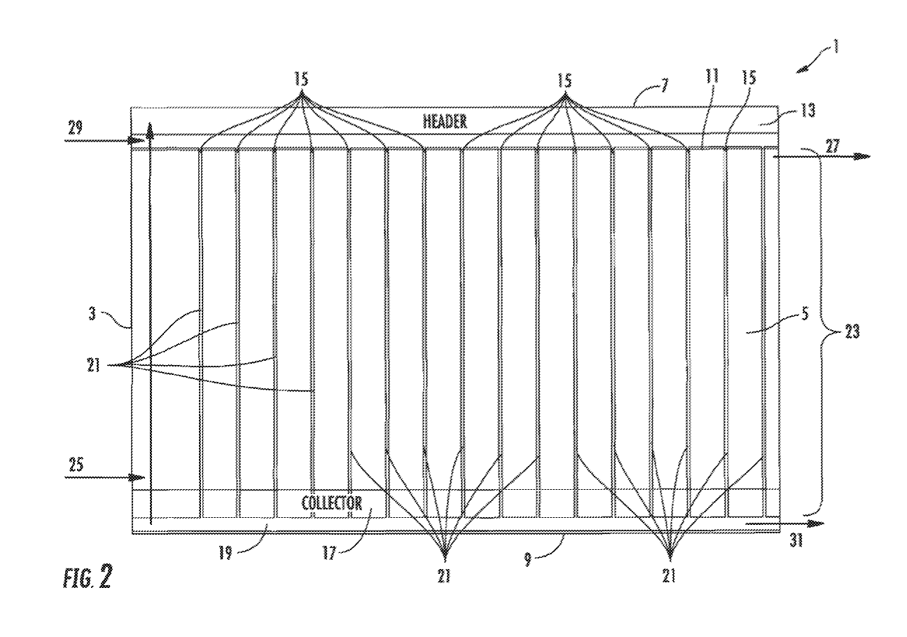

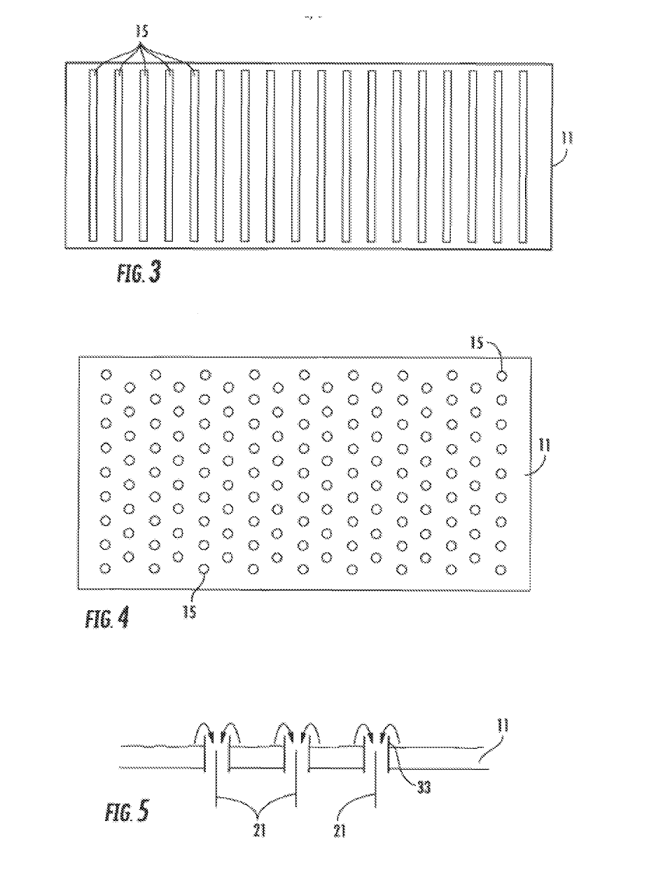

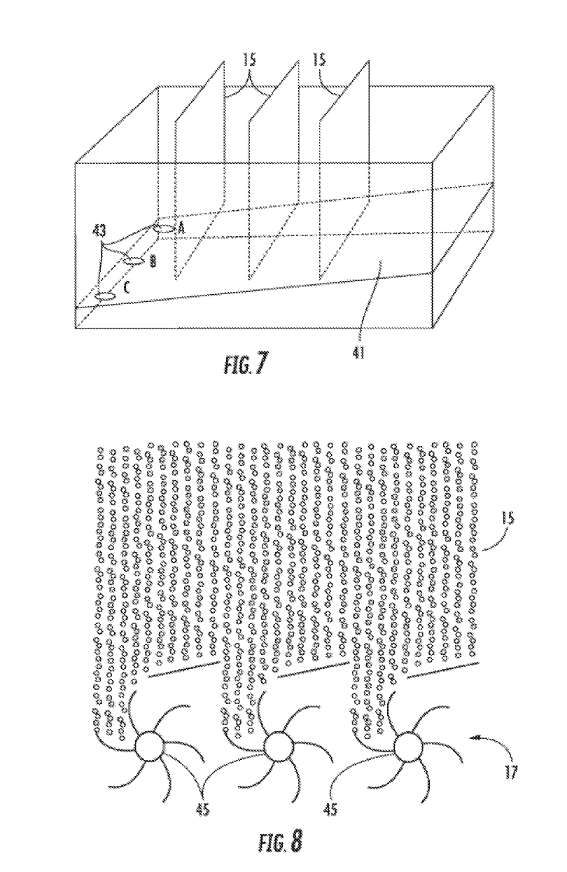

It is to be understood that the methods and apparatuses of the present invention may relate to features illustrated in one of the figures (or described by reference to that figure) in combination with features illustrated in another of the figures (or described by reference to that figure. For example, the invention may encompass a culture vessel as illustrated in FIG. 1 and FIG. 2 further comprising a header arrangement as illustrated in FIG. 3 or FIG. 4 or FIG. 6 and optionally further comprising the header feature illustrated in FIG. 5, and/or further features such as the feeder port, collection port, gas inlet and gas outlet illustrated in FIG. 5, and/or further comprising the collector plate and turbine features illustrated, respectively in FIG. 6 and FIG. 7.

### DETAILED DESCRIPTION OF THE EMBODIMENTS

General

A relatively low cost, highly productive culture vessel and method for growing photosynthetic organisms (in particular autotrophic algae) is provided. The culture vessel and method is primarily described in terms of a closed photobioreactor—however, the invention is not necessarily so limited. The culture vessel can be constructed in a way, and the method can be performed in a way, that is not strictly a “closed” system or “open” system.

Apparatus

One aspect of the invention is a culture vessel. The culture vessel can be a fully closed system that is not open to the outside environment. Alternatively, the culture vessel can be a partially open system that is at least partially open to the outside environment at one or more of the top or sides. The culture vessel can comprise a number of components.

A first component of the culture vessel is a partially or fully transparent or translucent enclosure. Preferably, the enclosure can be partially or fully transparent. More preferably, the enclosure can be fully transparent. Accordingly, the enclosure is preferably formed or constructed from a transparent material such as glass or plastic. As stated, the enclosure may be partially open to the environment or, alternatively, fully closed.

A second component of the culture vessel is a header, distribution plate, or distribution tubing (collectively, “header” unless otherwise expressly indicated) positioned within the upper portion of the enclosure with some degree of separation from the top of the enclosure (e.g., the inner surface of the ceiling if a ceiling is present). Preferably, the header can run roughly perpendicular to the top of the enclosure. Preferably, the header can be partially or fully transparent and constructed of the same material as the enclosure. Preferably, the height of the region above the header can represent at most 20%, more preferably at most 15%, and ideally at most 10%, of the height of the entire enclosure.

A third component of the culture vessel is one or more (preferably multiple) apertures located in, and penetrating through, the header. The shape of the apertures is not particularly important as long as the size and shape are sufficient to cause media flowing from the header through the apertures to take on the shape of narrow streams, sheets, or droplets akin to falling rain or thin waterfall like sheets. For example, the apertures may be in the form of small circular or oval shaped holes or, alternatively, thin elongated slots. Accordingly, in one aspect, the apertures in the header can include multiple slots sized so that liquid draining from the apertures form a thin sheet. Preferably, these slots can be parallel slots, and each slot can be substantially equidistant from an adjacent slot. Alternatively, or in addition, the apertures in the header may include multiple circular or oval shaped holes sized so that liquid draining from the apertures form droplets. Preferably, the holes can form an array wherein each hole is substantially equidistant from an adjacent hole. Regardless of shape, the dimensions of the narrowest part of the apertures can be from about 0.5 mm to about 5 mm, so that the algae suspension does not drain too fast or too slow. The aim is to size the apertures so that the resulting curtain has a relatively large surface area as compared to the volume.

The exact number of apertures can vary and can advantageously be selected to maximize light exposure. In other words, to a point, the addition of more steams, sheets, or droplets formed by the apertures can increase photosynthetic efficiency. Thereafter, the addition of more streams, sheets, or droplets can cause shading, which can interfere with photosynthetic efficiency. For any given shape and size of aperture, the maximum photosynthetic efficiency can rarely be reached until at least 20% (and typically much more than 20%) of the total surface area of the header is aperture.

A fourth component of the culture vessel is a collector section or pool positioned in, or formed largely or entirely by, the bottom of the enclosure. The pool can catch media flowing through the header. Preferably, the height of the collector section can represent at most 20%, more preferably at most 15%, and ideally at least 10%, of the height of the entire enclosure.

A fifth component of the culture vessel is a main chamber positioned between the header and collector section. This main chamber is typically not a separate structure but rather the intermediate region of the enclosure located between the header and the collector section. Preferably, the height of the main chamber can represent at least 60%, more preferably at least 70%, and ideally at least 80%, of the height of the entire enclosure.

Media flowing from the apertures in the header can move downward through the main chamber to the collector section. In one aspect of the invention, the media can fall freely through the main chamber as one or more streams, sheets, or droplets. In this embodiment the media in the chamber can behave much like falling rain and/or waterfalls. Alternatively, the media may descend through the main chamber along strings, wires, sheets, or mesh that hang(s) from each aperture (generally one per aperture) through a portion, preferably the majority, and ideally the entirety, of the main chamber. These strings, wires, sheets, or meshes can slow the rate of media descent through surface tension and, thereby, can increase residence time of the descending media in the main chamber. Among other things, increased residence time can increase the amount of light exposure and gas exchange per cycle, thereby reducing the number of cycles required, thereby reducing total pumping load (i.e., energy required for circulation) and associated cost.

In operation, a photosynthetic organism (preferably microalgae or cyanobacteria) in a liquid growth medium (preferably water or nutrient enriched water) can be circulated to the header, can flow through the apertures, and can descend as one or more (preferably multiple) narrow streams, sheets, or droplets through the main chamber toward the collector section. Preferably, the media can thereafter be re-circulated to the header at the top of the enclosure (e.g., by an internal or external pump, air lift, or other conventional means). The process may be repeated multiple times until a desired growth density of the organism in the media is achieved. The organism may, thereafter, be harvested either continuously or in batch fashion from the media captured in the collector.

Optionally, an inclined collector plate can also be positioned above the collector section. The collector plate can be a substantially planar piece containing apertures that drain to the pool of algae suspension below. The collector plate can preferably be sloped so that the media drains into the pool. The sloped collector can allow maximum/increased drainage of the algae suspension for circulation. It can additionally or alternately ensure that all of the algae suspension can be recycled with little stagnation. Among other things, this can improve the uniformity of the process conditions that the algae experience (e.g., light exposure, gas exchange, temperature, pH, etc.).

Optionally, one or more turbines can also be included. The turbines can convert kinetic energy from the falling media (e.g., water or nutrient enriched water) into electricity. The falling media for the turbines can be from the apertures in the header. Alternatively, or in addition, the falling media for the turbines can be from the apertures in a collector plate if a collector plate is used. The electricity can then be used to power a pump or one or more other devices to recirculate media from the pool to the header, thereby reducing the total energy needs and cost of the system.

The culture vessel may also include other components typical in a closed and/or open PBR. Typical components include gas inlet(s), gas outlet(s), feed port(s), harvesting port(s) and, optionally, one or more heat management components.

FIGS. 1 and 2 illustrate a preferred embodiment of a culture vessel constructed in accordance with the invention. The culture vessel 1 in FIG. 1 shows the main components of the invention. Many of the features may be altered or reconfigured or even deleted and still be effective as described in the additional examples.

The culture vessel 1 of FIG. 1 contains a transparent enclosure 3 with sides or walls 5, a ceiling 7 and a floor 9. The transparent enclosure 3 shown is substantially rectangular with right angles at the four corners. The shape of the enclosure 3 however is not critical and can be any shape. The enclosure 3 is preferably entirely transparent to allow light to pass through at all sides 5 and the ceiling 7. Alternatively, or in addition, one or more of the ceiling 7 or sides 5 can be partially open to the environment.

The culture vessel 1 has a header or dispersion plate 11 near, but below, the ceiling of the transparent enclosure. The header 11 is generally a planar piece of material that extends substantially perpendicular from the sides 5 of the enclosure 3. The enclosure contains head space 13 between the header 11 and the ceiling 7 of the enclosure 3. The header 11 contains at least one aperture 15 but preferably a plurality of aperture 15. The shape and positioning of the apertures 15 is discussed in more detail below.

On or near the floor or bottom 9 of the enclosure 3 is a collector section 17. In FIG. 1, the collector section 17 is not a separate component but rather a section at the bottom 9 of the enclosure 3 onto which a liquid media containing a photosynthetic organism (assumed, in discussion of the figures, to be a suspension of algae in nutrient enriched water) falls. Preferably, a shallow pool 19 of the algae suspension is maintained in the collector section 17 to minimize the impact and splashing of media from the header 11. The falling algae suspension descends in the form of curtains, sheets, thin films, droplets, etc. (collectively, liquid curtains) 21 to reach the collector section 17.

Between the header 11 and collector section 17 is the main chamber 23. The liquid curtains 21 fall between the header 11 and the collector section 17 after the algae suspension drains through the apertures 15 in the header 11. The algae suspension drains down from the apertures 15 forming an array of liquid curtains 21. The liquid curtains function as thin flat panels which yield high productivity and high concentration due to (i) light dilution and (ii) short light path. The gas exchange occurs along the liquid-gas interface as the liquid curtains flow down. The dissolved oxygen is stripped out and the algae suspension, in turn, is enriched with CO₂. Preferably, the algae suspension is then recirculated from the collector section 17 back up to the head space 13. The algae suspension is recirculated via a recirculation pathway 24, which can be inside or outside the enclosure 3. Preferably, a low shear pump or an air lift is used to transport the algae suspension up to the head space 13. The air lift can utilize compressed air or any other gas (including C₂ and/or other nutrient gas).

The culture vessel 1 contains other features typically found in closed and/or open PBRs. For example, the vessel contains one or more gas inlet(s) 25 and one or more gas outlet(s) 27. The vessel also contains one or more feed port(s) 29 and one or more harvesting port(s) 31. Optionally, a heat exchange component (not shown) to remove heat may be included.

With regard to the transparent enclosure 3, the materials used to construct the sides or walls 5 and ceiling 7 of enclosure are preferably completely transparent to either visible light or photosynthetically active radiation (PAR). The materials used to make the enclosure 3 should preferably be the cheapest, robust, transparent materials that do not absorb or modify sunlight in the PAR frequency. Optionally, IR-reflective coatings on the materials can also be used on the walls 5 or ceiling 7. IR reflective coatings may be beneficial for the vessel 1 as the coating will reduce the rate of heating within the enclosure 3.

The enclosure 3 functions to contain the algae suspension and separate the algae suspension from the outside environment. This functions to significantly reduce contamination by predators or other algae species. The enclosure 3 may be constructed of pliable or rigid materials such as polypropylene or poly(methyl methacrylate) for example (also known as PMMA, acrylic glass, or Plexiglass™). If the enclosure is a closed system, then, in operation, the enclosure will be filled by injecting an appropriate gas mixture (for example air+1-3% CO₂ or flue gas). Once filled, the rate of gas input to the enclosure should be maintained at the same rate as gas output.

With regard to the header 11, the head space 13 may be constructed by partitioning the top section of the enclosure 3 with a planar piece of plastic. The planar piece of plastic may be rigid or flexible so long as it is sufficiently supported to maintain a flat or substantially flat configuration. This layer of plastic needs to be sturdy enough to withstand some liquid accumulation across the top surface area of the header 11 without deforming its shape (to avoid pooling). The planar piece may be integral with the walls 5 of the enclosure or a separately connected piece.

Similarly, the entire vessel 1 can be of unitary construction or it can be completely modular, or some combination thereof For example, the header 11 and head space 13 and top of the enclosure 7 can be a modular component that is placed on the main chamber 23.

The header contains one or more apertures 15. FIGS. 3-6 show several views of different header embodiments. FIG. 3 shows the top view of header 11 where the apertures 15 are a series of parallel slots. Slots will produce thin films when the algae suspension drains through the apertures, such as shown in FIGS. 1 and 2. The number, the size, the distance between two slots, and the volumetric flow rates and even the shape can each be varied according to the requirements of the algae. All of these parameters dictate the degree of light dilution in the resulting thin film, sheet, droplet, etc., which in turn influences the biomass productivity and concentration of algae in the media. The resulting geometry of the liquid curtain 21 does not need to be rectangular; it can be cylindrical as welt. The liquid curtain 21 is a function of the size of the main chamber 23 dimensions and the aperture 15 size and shape. As a result, the liquid curtain 21 can be short and wide (as a rectangular prism), or it can be tall and narrow (such as a cylinder).

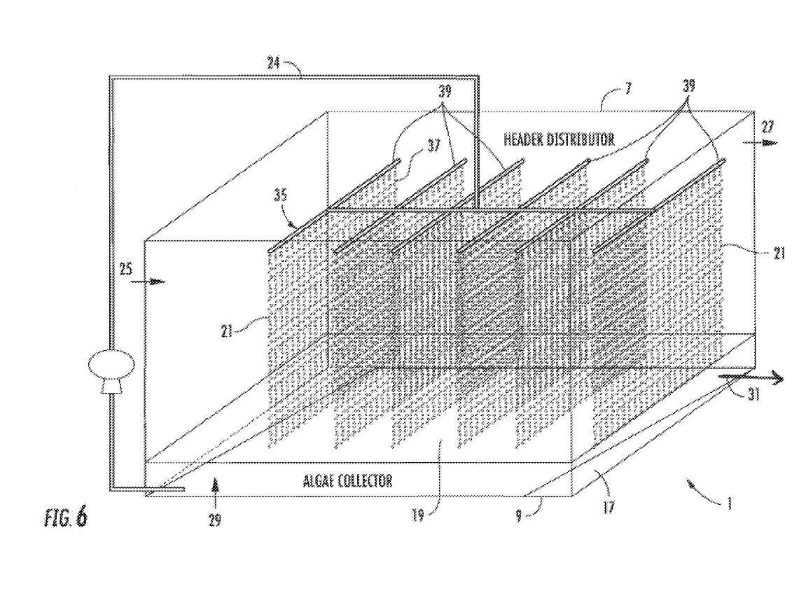

The apertures 15 can be any shape or configuration such as slits (i.e., rectangular holes) to form sheets or films of waterfall, or circular holes to form droplets, jets or streams, Such a configuration may result in increased surface area in the liquid curtain 21, which promotes light dilution, a short light path, and increased surface area for as exchange. The top view of a header 11 with rectangular slits 15 is shown in FIG. 3. The top view of a header 11 with circular holes 15 is shown in FIGS. 4. The circular holes 15 will produce a jet or stream of algae suspension, Although the dimensions may vary, the narrowest part of the apertures will preferably be from about 0.5 to about 5 mm so that the algae suspension does not drain too fast or too slow. The aim is to size the apertures so that the resulting curtain has a relatively large surface area as compared to the volume.

FIG. 5 illustrates a side view of an optional feature of the header 11. The apertures 15 have lip or raised edge 33 surrounding the aperture 15, The raised edge 33 is raised above the height of the surface of the header 11. In this way, the liquid media will fill in around all the apertures 15 before spilling into any of the apertures 15. This is especially important if the recirculating path is located at one end of the enclosure 1. In this way, it is possible to ensure that the liquid media spills in all of the apertures 15 and not just the apertures nearest the recirculating pathway.

With regard to the collector section 17, it is preferably a pool of algae suspension at the bottom of the enclosure. Having the liquid curtains 21 fall into a pool of algae suspension at the bottom minimizes the impact and splashing of the liquid curtains 21 as they reach the bottom. This collector section 17 also serves as reserve volume of algae suspension to replenish the algae suspension in the head space 13 via the recirculation pathway.

FIG. 6 illustrates an alternate embodiment of the culture vessel I of the invention. The culture vessel has a transparent enclosure 3 with sides 5, a ceiling 7, and a floor 9. However, instead of a plate like header 11 with apertures 15 there is a tubular header (hereinafter distributor) 35. The distributor 35 is constructed of a set of tubing. The tubing itself has apertures (not shown) through which the liquid media drains. The apertures may be any variety as described above. In this instance, the tubing has circular apertures to produce droplets. Further, not all of the tubing need have apertures. In this instance there is main tube 37 from which side tubes 39 extend on either side. The main tube 37 received liquid media directly from the recirculation pathway 24. The tubes 37 and 39 that make up the distributor 35 are preferably, but not necessarily, transparent.

FIG. 7 shows an alternate embodiment of the collector section 17. In this embodiment the liquid curtains 15 drain through the main chamber 23 to hit a collector plate 41. The collector plate 41 is a substantially planar piece near the floor 9 of the enclosure 3. The collector plate 41 has apertures 43 which drain to the pool 19 of algae suspension below. The collector plate 41 is preferably sloped so that the liquid media drains into the pool 19 below. The sloped collector allows maximum drainage of the algae suspension for circulation. It also ensures that all of the algae suspension can be recycled with little stagnation.

FIG. 8 shows a further variation of the collector section 17 that includes one or more turbines above the pool 19 (or collector plate 41 if there is one). The turbine or turbines 45 convert the kinetic energy of the falling liquid media into electricity. The electricity can then be used to power the pump or one or more other devices that recirculate the algae suspension from the pool 19 to the header or distributor 35, thereby offsetting some portion of the power required to recirculate the algae suspension from the collector to the header.

In operation, the composition of the gas entering the enclosure can be a function of the pH of the algae suspension or kept constant. This can be determined by the requirements of the specific algae. In a closed system, the gas pressure can typically be kept at atmospheric as much as possible and should preferably have low momentum as it enters the enclosure. This can minimize momentum interaction with the liquid curtains 21. Once the enclosure is filled with gas, the algae inoculum suspension and media can be injected into the enclosure through the feed port(s) 29 in the head space 13. The algae suspension can be harvested via the harvesting port(s) 31 in the collector section 17.

A heat exchange component can typically be used in operation. The heat exchange component can be internal or external but an external heat exchange component can be preferable in most instances. An external heat exchange component is likely easier to construct and less likely to interfere with the function of the vessel 1. Some illustrative examples of suitable heat exchange components include heat exchangers, water sprays, and passive heating/cooling materials. Alternatively, use of a hot or cold gas to maintain constant algae suspension temperature may be employed. In this way, the temperature of the algae suspension can be controlled by adjusting the flow rates of the gas and/or the mixture of hot/cold gas.

In addition it can be preferable that all of the components are transparent. This can enable the algae to grow throughout the photobioreactor and not just in main chamber 23.

The culture vessel can be operated in a batch mode or a continuous mode. Harvesting the algae may be performed using the techniques employed with any other open and/or closed PBR system.

Method

Another aspect of the invention is a method for growing photosynthetic organisms. The method may be employed in a fully closed system. Alternatively, the method may be employed in a partially closed system. The method can comprise a number of steps. The first step can be providing nutrients to a media containing a photosynthetic organism, The second step can be circulating the media to a header. The third step can be forming the media into one or more descending streams, sheets, or droplets (collectively, liquid curtains). This step can typically be accomplished by multiple apertures in the header. Although the dimensions of the apertures may vary, the narrowest part of the apertures can preferably be from about 0.5 mm to about 5 mm, so that the algae suspension does not drain too fast or too slow. The liquid curtains of media can then free fall or, alternatively, travel downward along a string, wire sheet or mesh. The fourth step can be capturing the descending media in a collector. The fifth step can be re-circulating the media from the collector to the header, preferably using a pump or air-lift. During the process, the media can be exposed to light, preferably from the sun. Ideally, steps two through five can be repeated multiple times until a target organism growth density is achieved.

Preferably, the method can employ the apparatus previously described. Accordingly, any of the other optional and/or preferable features of the apparatus may be employed in the method. For instance, the method may employ an inclined collector plate positioned above the collector, and/or one or more turbines positioned above the collector, and/or other typical PBR components such as gas inlet(s), gas outlet(s), feed port(s), harvesting port(s), and one or more heat management components.

Photosynthetic Organisms

All of the apparatuses and methods described herein can be used with any type of relatively small photosynthetic organism but can work especially well with micro-organisms, such as algae (i.e., microalgae and cyanobacteria), that require intermittent diffuse light to achieve optimal growth. Examples of suitable algae can include rhodophytes, chlorophytes, heterokontophytes, tribophytes, glaucophytes, chlorarachniophytes, euglenoids, haptophytes, cryptornonads, dinoflagellums, phytoplanktons, and the like, and any combination thereof. In one embodiment, algae can be selected from the classes Chlorophyceae and/or Haptophyta. Specific species for use in the invention can include, but are not limited to, Neochlorisoleoabundans, Scenedesmusdimorphus, Euglena gracilis, Phaeodactylumtricornutum, Pleurochrysiscarterae, Prymnesiumparvum, Tetraselmischui, and Chlamydomonasreinhardtii. Additional or alternate algal sources can include one or more microalgae of the Achnanthes Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Platymonas, Pleurochrysis, Pleurococcus, Prototheca, Pseudochlorella, Pyramimonas, Pyrobotrys, Scenedesmus, Skeletonema, Spyrogyra, Stichococcus, Tetraselmis, Thalassiosira, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, lyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Totypothrix, Trichodesmium, Tychonema, and Xenococcus species.

Potential Benefits

All of the apparatuses and methods described herein can provide one or a number of significant advantages compared to conventional growth systems (e.g., open ponds and closed PBR systems). These potential advantages are set forth below.

First, the continuously flowing, free falling, thin streams, sheets or droplets of media can provide interfaces for natural gas exchange (e.g., CO₂ enrichment to the algae suspension and O₂ stripping from the algae suspension). The inventive culture vessels and methods can provide the same light dilution and short tight path as an array of flat panel PBRs. As a result, comparatively higher organism concentrations and/or growth rates can be achieved. For example, algae concentrations of 10,000 to 30,000 ppm and, possibly, higher, can be achieved as well as densities of 10-30 g/L and, possibly higher. Additionally or alternatively, algae productivities of at least 10 g/L (AFDW), and optionally up to 20 g/L, (AFDW), can be achieved. Further additionally or alternatively, average algae growth rates (over at least 3 days, alternately over at least 4 days) of at least 1.2 g/L/day (AFDW), e.g., at least 1.4 g/L/day (AFDW), at least 1.5 g/L/day (AFDW), at least 1.6 g/L/day (AFDW), at least 1.7 g/L/day (AFDW), at least 1.8 g/L/day (AFDW), at least 1.9 g/L/day (AFDW), or at least 2 g/L/day (AFDW), and optionally up to 5 g/L/day (AFDW) or up to 4 g/L/day (AFDW), can be achieved. The inventive culture vessels and methods can provide 20-100 times or more algae concentration than a typical raceway pond. This, in turn, can result in a cost savings during the growth process (because less water or other liquid media may be needed) and/or downstream algae harvesting operations (because less water or other liquid media may need to be separated and/or recycled).

Second, in contrast to the relatively high shear experienced by the organism suspensions in a typical short-light path PBR, the free falling organism suspension grown using the invention can advantageously experience relatively low shear. Low shear can reduce the probability of cell breakage. Accordingly, the invention may be especially suitable for use with algae species (and other organisms) that are relatively more sensitive to hydrodynamic stress. More particularly, the invention may be especially suitable for use with species that are not able to thrive in other/standard tubular photobioreactors.

Third, because of the unique structure of the vessel, where the media in the main chamber is free falling or descending on the exterior of a thread or sheet, there can be a substantial absence of biofouling along the photobioreactor surfaces where sunlight impinges the algae suspension. Biofouling can still occur within the header or collector sections, but should not typically occur inside the main chamber, at least not at relatively quick time scales. The reduced biofouling can result in lower maintenance/cleaning requirements and associated costs.

Fourth, the apparatuses of the invention can be more readily and/or cheaply mass produced, installed, and/or scaled up than currently known growth systems. Reduced material and reduced support structure may be needed to construct the vessels, making the inventive culture vessels potentially orders of magnitude cheaper to construct than flat panels reactors. Very large culture vessels can be possible (e.g., 20 feet by 100 feet). The inventive culture vessels can require minimal ground preparation and may not need the precise grading of a raceway pond.

Illustrative Embodiments

Embodiment 1 A culture vessel for growing a photosynthetic organism comprising: (i) a partially or fully closed enclosure that is at least partially transparent or translucent; (ii) a header positioned in the upper portion of the enclosure; (iii) one or more apertures in the header; (iv) a collector section positioned in the bottom portion of the enclosure; and (v) a main chamber positioned between the header and collector section, wherein a photosynthetic organism in a liquid growth media is circulated to the header, flows through the apertures, and then descends as a one or more streams, sheets or droplets through the main chamber toward the collector.

Embodiment 2. The culture vessel of embodiment 1 wherein the vessel is a fully closed system that is not open to the outside environment.

Embodiment 3. The culture vessel of embodiment 1 wherein the vessel is a partially open system that is open to the outside environment at one or more of its top and sides.

Embodiment 4. The culture vessel of any one of the previous embodiments wherein the liquid growth media is water or nutrient enriched water.

Embodiment 5. The culture vessel of any one of the previous embodiments wherein the organism is selected from microalgae and cyanobacteria.

Embodiment 6. The culture vessel of any one of the previous embodiments wherein the enclosure is partially or fully transparent.

Embodiment 7. The culture vessel of any one of the previous embodiments comprising multiple apertures, thereby causing the media to flow through the main chamber as multiple streams, sheets or droplets.

Embodiment 8, The culture vessel of any one of embodiments 1-6 wherein the apertures in the header include multiple slots sized an that liquid draining from the apertures form a thin sheet.

Embodiment 9. The culture vessel of any one of embodiments 1-6 wherein the apertures in the header include a series of parallel slots and wherein each slot is substantially equidistant from the adjacent slot.

Embodiment 11. The culture vessel of any one of embodiments 1-6 wherein the apertures in the header include multiple circular or oval shaped holes sized so that liquid draining from the apertures form droplets.

Embodiment 12. The culture vessel of any one of embodiments 1-6 wherein the apertures in the header include an array of circular or oval shaped holes and wherein each hole is substantially equidistant from the adjacent hole.

Embodiment 13. The culture vessel of any one of embodiments 1-6 wherein the descending media falls freely as a one or more streams, sheets, or droplets through the main chamber into the collector.

Embodiment 14. The culture vessel of any one of the previous embodiments wherein the descending media travels downward along a string, wire, sheet, or mesh that hangs from each aperture to slow the rate of media decent and, thereby, increase the residence time of the descending media in the main chamber.

Embodiment 15. The culture vessel of any one of the previous embodiments wherein the dimensions of the narrowest part of the apertures is preferably from about 0.5 mm to about 5 ram so that the media does not drain too fast or too slow,

Embodiment 16. The culture vessel of any one of the previous embodiments wherein the me a is re-circulated from the collector to the header using a pump or air-lift.

Embodiment 17. The culture vessel of any one of the previous embodiments further comprising an inclined collector plate positioned above the collector section.

Embodiment 18. The culture vessel of any one of the previous embodiments further comprising one or more turbines above the collector section.

Embodiment 19. The culture vessel of any one of the previous embodiments further comprising gas inlets and outlets, feed and harvesting ports, and heat management components.

Embodiment 20. A method for growing a photosynthetic organism comprising the following steps: (1) providing nutrients to a media containing a photosynthetic organism; (ii) circulating the media to a header; (iii) forming the media into one or more descending streams, sheets or droplets; (iv) capturing the descending media in a collector; and (v) re-circulating the media from the collector to the header.

Embodiment 21. The method of embodiment 20 wherein the step of forming the media into one or more descending streams, sheets, or droplets is accomplished by multiple apertures in the header.

Embodiment 22. The method of embodiment 20 or 21 wherein the header and collector are two parts of a fully or partially enclosed system.

Embodiment 23. The method of any one of embodiments 20-22 wherein the descending media is exposed to light from the sun.

Embodiment 24. The method of any one of embodiments 20-23 wherein the dimensions of the narrowest part of the apertures is preferably from about 0.5 mm to about 5 mm so that the media does not drain too fast or too slow.

Embodiment 25. The method of any one of embodiments 20-24 wherein the descending streams, sheets or droplets of media free fall.

Embodiment 26. The method of any one of embodiments 20-25 wherein the descending streams, sheets or droplets of media travel downward along a string, wire, sheet, or mesh.

Embodiment 27. The method of any one of embodiments 20-26 wherein the media is re-circulated from the collector to the header using a pump or air-lift.

Embodiment 28. The method of any one of embodiments 20-27 further comprising providing an inclined collector plate positioned above the collector.

Embodiment 29. The method of any one of embodiments 20-28 further comprising providing one or more turbines above the collector.

Embodiment 30. The method of any one of embodiments 20-29 further wherein steps (ii) through (v) are repeated multiple times until a target organism growth density in the media is achieved.

Alternatives

There will be various modifications, adjustments, and applications of the disclosed invention that will be apparent to those of skill in the art, and the present application is intended to cover such embodiments. Accordingly, while the present invention has been described in the context of certain preferred embodiments, it is intended that the full scope of the invention be measured by reference to the scope of the following

EXAMPLES

Examples 1-4

Two bench-scale rain-effect photobioreactors were built to assess technical feasibility of the rain-effect photobioreactor (PBR), The PBRs were operated with ˜1-1.5 L of algae culture volume and used shower-like header design combined with a peristaltic pump for recirculation. In total, four batch runs were completed under constant illumination with artificial white light source and using wild type (wt.) Tetraselmis algae strain. The experiments demonstrated robust operation with culture densities reaching about 13 g/L (1.3 wt%) ash-free dry weight (AFDW) within ˜10 days under constant and continuous illumination, Highest productivity levels observed during the experiments were about 2 g/L/day AFDW at culture densities greater than about 1 g/L AFDW, suggesting a good potential from growth and harvesting perspectives.

The design of the bench-scale rain-effect (waterfall effect) PBRs is based on the idea of combining multiple flat-panel PBRs into a single, low-cost unit, where algae culture is circulated between bottom and top reservoir via pumps or airlift. The culture exiting the top reservoir can be dispensed in the form of parallel waterfalls or spray droplets. The surface tension acting on the exiting waterfall films can create even thinner films than those achievable in flat panel PBRs, enabling further decrease of the light pathway thickness and potentially further increase in maximum productivity and culture density. Furthermore, countercurrent flow of gases injected directly into the headspace of the PBR can enable more efficient gas exchange, allowing better CO₂ delivery and O₂ removal. Airlift circulation of culture through pipes with an air/CO₂ mixture can further enhance gas delivery by direct introduction into the liquid stream.

The two bench-scale prototype units have been constructed to operate with peristaltic pumps. The PBRs were used with a perforated plate header design to allow the algae culture to discharge in the form of jets with breakup and droplets. Four experimental runs were completed with wt. Tetraselmis under 24-hour illumination with white-tight fluorescent light source in batch mode. The experiments showed rapid growth of the algae culture, reaching a maximum of about 9 g/L AFDW biomass density within about 8 days and, under another set of conditions, reaching about 13 g/L AFDW after about 20 days, both at ˜1,200 μE/m²/sec illumination (1 μE=1 micro “Einstein”≈1 μmole (˜6.022*10¹⁷) of photons in a 400-700 rim wavelength range). The maximum productivity observed was about 2/g/L/day showing further potential of the system to achieve high culture density and productivity. Note that the biomass productivity here is described as volumetric productivity, and not areal productivity, due to the difficulty of defining the area for a small bench-scale PBR unit.

The rain-effect PBR small-scale units have been constructed from Plexiglass™ having >95% optical transmittance in the photosynthetically active radiation (PAR, 400-700 nm) range of light frequencies. For the initial assessment, the units were built with a top header containing an array of holes through which circulated culture could exit in the form of trickles or droplets. In the first design, there were ˜145 holes, about 2 mm diameter, allowing for a minimum circulation flow rate of ˜6 L/min. The size of holes was subsequently increased to about 3 mm each, because the holes were observed to be clogging with algae at higher culture concentrations. During the initial experiment, air mixed with CO₂ was directly delivered to the PBRs through ˜0.2 μm. PTFE filters at a flow rate of ˜30 sL/hour (sL=standard Liter−a liter of gas at standard conditions of ˜1 atm and ˜25° C.). Following the first experimental run, to decrease the amount of evaporative water losses, the air/CO₂ mixture was setup to first saturate by sparging with DI water before entering the PBR. The second unit was constructed with a doubled number of holes (i.e., ˜290, all 3 mm in diameter) and was operated at ˜15 L/min circulation flow rate. Additionally, after the first experiment, a sampling T-valve port was installed in the pump tubing to minimize external contamination. Before each run, the PBR was filled with DI water/bleach solution and allowed to circulate the solution for at least ˜30 minutes to a maximum of ˜18 hours (overnight), The tank was then rinsed 3 times with fresh DI water and finally sprayed with ˜70% ethanol/water mixture. The tank was allowed to thoroughly dry before use.

A It experiments were carried out with wt. Tetraselmis algae strain. The media was made with ˜4 wt% of Instant Ocean. In addition, the nutrient make-up for the first 3 sets of experiments was comprised of standard lx concentration of the ProLine F/2 A (minerals) and ProLine F/2 B (vitamins), i.e., about 1.3 ml/L for both components (which are commercially available, e.g., from Aquatic Eco Systems Inc. of Apopka, Fla.). In the fourth experiment, the media concentration was increased to 2x, Le., about 2.6 ml/L for both components. In the first two experiments, the initial culture volume was ˜1 L, while in the last two the volume was ˜1.5 L. Samples, ˜50 mL, were withdrawn daily during weekdays, and the culture was replenished with ˜50 mL of fresh media. Analytical measurements performed included AFDW, optical density (OD), pulse-amplitude modulated fluorescence (PAM), and optical microscopy.

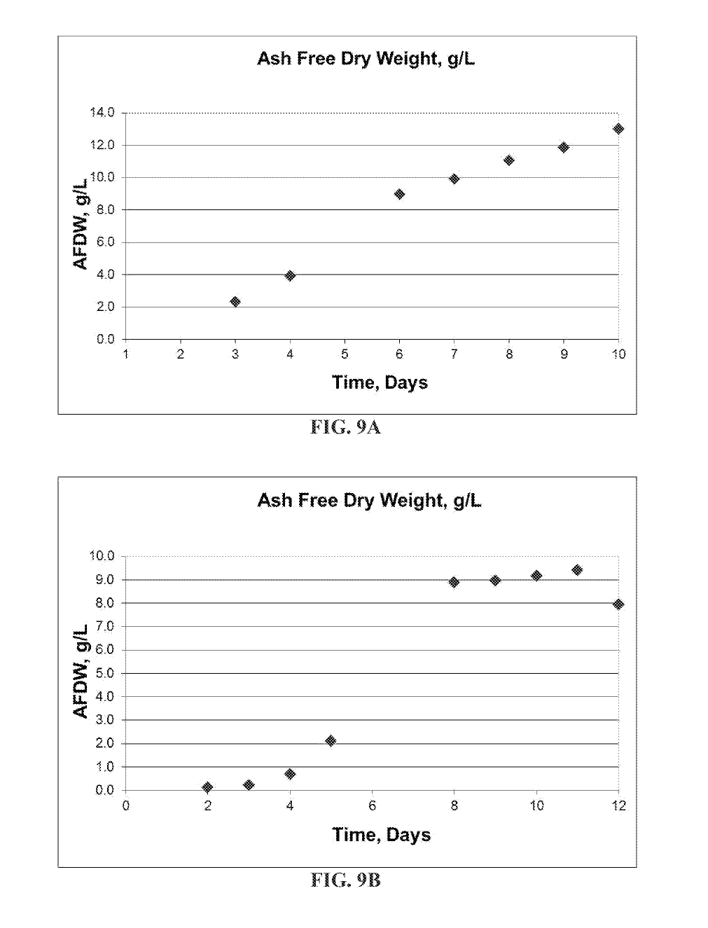

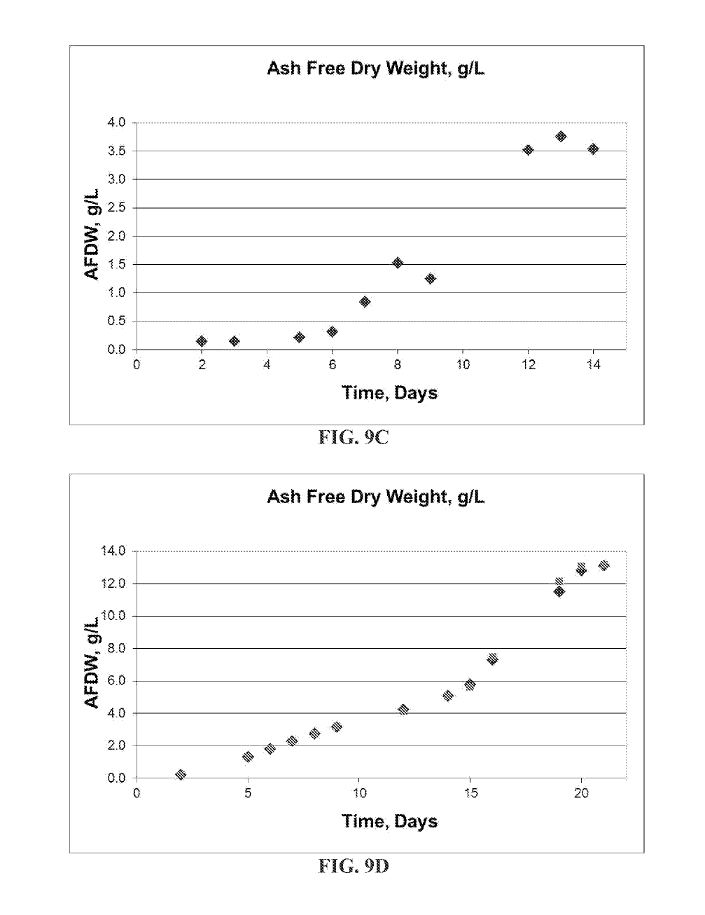

The first experiment was performed with constant light illumination and gas flow rate. The light used had an approximate average intensity of ˜1,200 μE/m²/sec. The circulation flow rate was ˜6 L/min. FIG. 9A shows the growth curve in terms of the AFDW measurements. Inspection of the culture showed significant evaporation of water due to large volumetric flow rate of the dry gas. At the end of the ˜10 days, about ⅔ of the water volume was lost. As such, the observed algae concentration was artificially increased. Additionally, it was observed that, at the high end of the concentrations, the ˜2 mm openings appeared to be clogging with algae precipitates. Important to note is that, at this circulation flow rate, the culture temperature remained relatively steady throughout the entire run at ˜25° C. Without being bound by theory, the increase above room temperature was believed to primarily be a result of frictional heating at the head of the peristaltic pump and not due to the light absorption. This speculation was further substantiated with subsequent experiments, where two pumps were used for circulation and where temperature had stabilized at ˜30° C although the same light intensity was used. The pH of the culture remained at ˜7.5 for the entire duration of the run, indicating relatively efficient gas delivery through the countercurrent gas flow. The samples showed significant presence of bacterial growth at the end of the run, prompting design changes to more effectively isolate the system from the surrounding environment.

To improve the system operation, the incoming dry gas was set to first sparge through a sealed DI water reservoir in order to saturate the gas with water vapor. Water saturation appeared to solve the problem, resulting in minimal water losses in runs (Examples) #2-4. Additionally a sampling port was installed in the pumping line next to the bottom reservoir's outlet port, whereas in run (Example) #1 the sample was withdrawn from the top head compartment thus exposing the culture to external environment.

Repetition of the experimental run #1 with these design changes gave improved assessment of the performance. FIG. 9B shows the AFDW results of run #2, The results showed relatively rapid growth of the culture, reaching a maximum of about 9 g/L AFDW in just ˜8 days. The productivity between days 5 and 8 appeared to fall in the range of ˜2 g/L/day. was also observed that the cells changed morphology toward the end of the run, which may have been an indication of nutritional limitation. This speculation was explored in run (Example) #4. These results were promising and prompted further evaluation of the system,

To further evaluate the design at the bench-scale level a second unit was constructed with double the number of holes to increase light utilization and increase the circulation flow rate. The number of holes was increased to ˜290, allowing for doubling of the recirculation rate to ˜16 L/min. The increased circulation flow rate falls at approximately the maximum operational limit of the peristaltic pumps available for the experiment and, at this rate, substantial wear and rapid failure of the tubing was observed.

To increase the circulation flow rate while operating the pumps at a safe flow range, two identical pumps were run instead in parallel. Run #3 was also conducted with increased culture volume (i.e., ˜1.5 L) to compensate for increased dead volume in the tithing. Results (AFDW vs. time) of run #3 are shown in FIG. 9C. The results showed decreased maximum culture density and a maximum growth rate between days 6 and 12 of only ˜0.5 g/L/day. Investigation of the operating conditions showed that the temperature of the culture was increased to ˜30° C., which could potentially have had a significant impact on the growth kinetics. The increased temperature was attributed to doubling of the number of pumps, as it was the only variable changed from previous runs. Additionally, during the run, the ultra-pure water supply was discovered to have failing filters and of the media, as well as the culture, was found to be strongly acidic. All of these factors were presumed to have caused the negative impact on the run.

In a final run #4, the effect of increasing nutrient concentration on the growth characteristics was explored. The experiment was performed with gradually increasing light intensity to explore effect of slower adaptation of the culture to high light intensity. The nutrient concentration was doubled to ˜2.6 ml for both F/2 ProLine A and F/2 ProLine B. Initially, the light intensity was set at ˜400 μE/m²/sec, and, after reaching maximum concentration, the level was increased to the maximum of ˜1200 μE/m²/sec. The top header was replaced with the original, having ˜145 holes; however, the diameter of the holes was increased to ˜3 mm. The minimum circulation flow rate was also increased to ˜8 L/min with the use of a single pump. Results of the nm #4 are shown in FIG. 91).

As suspected, decreasing circulation flow rate decreased the culture temperature back to ˜25° C. Furthermore, both increasing the nutrient concentration and stepwise adaptation to the high light intensity proved to have beneficial effect on the maximum culture concentration. The maximum culture density reached about 13 g/L AFDW, and, importantly, the growth rate between ˜8 and ˜12 g/L AFDW was on the order of ˜1.5 g/L/day AFDW.

What is claimed is:

1. A culture vessel for growing a photosynthetic organism comprising:
(i) a partially open or fully closed enclosure that is at least partially transparent or translucent;
(ii) a header positioned in the upper portion of the enclosure;
(iii) one or more apertures in the header;
(iv) a collector section positioned in the bottom portion of the enclosure; and
(v) a main chamber positioned between the header and collector section,
wherein a photosynthetic organism in a liquid growth media is circulated to the header, flows through the apertures, and then descends as a one or more streams, sheets, or droplets through the main chamber towards the collector section.

2. The culture vessel of claim 1 wherein the vessel is a fully closed system that is not open to the outside environment.

3. The culture vessel of claim 1 wherein the vessel is a partially open system that is at least partially open to the outside environment at one or more of its top and sides.

4. The culture vessel of claim I wherein the liquid growth media is water or nutrient enriched water.

5. The culture vessel of claim 1 wherein the organism is selected from microalgae and cyanobacteria.

6. The culture vessel of claim 1 wherein the enclosure is partially or fully transparent.

7. The culture vessel of claim 1 comprising multiple apertures, thereby causing the media to flow through the main chamber as multiple streams, sheets, or droplets.

8. The culture vessel of claim 1 wherein the apertures in the header include multiple slots sized so that liquid draining from the apertures form a thin sheet.

9. The culture vessel of claim 1 wherein the apertures in the header include a series of parallel slots and wherein each slot is substantially equidistant from the adjacent slot.

11. The culture vessel of claim 1 wherein the apertures in the header include multiple circular or oval shaped holes sized so that liquid draining from the apertures form droplets.

12. The culture vessel of claim 1 wherein the apertures in the header include an array of circular or oval shaped holes and wherein each hole is substantially equidistant from the adjacent hole.

13. The culture vessel of claim 1 wherein the descending media falls freely as a one or more streams, sheets, or droplets through the main chamber into the collector section.

14. The culture vessel of claim 1 wherein the descending media travels downward along a string, wire, sheet, or mesh that hangs from each aperture and slows the rate of media decent, thereby increasing the residence time of the descending media in the main chamber.

15. The culture vessel of claim 1, wherein the dimensions of the narrowest part of the apertures is from about 0.5 to about 5 mm so that the media does not drain too fast or too slow.

16. The culture vessel of claim 1 wherein the media is re-circulated from the collector section to the header using a pump or air-lift.

17. The culture vessel of claim 1 further comprising an inclined collector plate positioned above the collector section.

18. The culture vessel of claim 1 further comprising one or more turbines above the collector section.

19. The culture vessel of claim 1 further comprising as inlets and outlets, feed and harvesting ports, and heat management components.

20. A method for growing a photosynthetic organism comprising the following steps:
(i) providing nutrients to a media containing a photosynthetic organism;
(ii) circulating the media to a header;
(iii) forming the media into one or more descending streams, sheets or droplets;
(iv) capturing the descending media in a collector; and
(v) re-circulating the media from the collector to the header.

21. The method of claim 20 wherein the step of forming the media into one or more descending streams, sheets or droplets is accomplished by multiple apertures in the header.

22. The method of claim 20 wherein the header and collector are two parts of a fully or partially enclosed system.

23. The method of claim 20 wherein the descending media is exposed to light from the sun.

24. The method of claim 21 wherein he dimensions of the narrowest part of the apertures is preferably from about 0.5 to about 5 mm so that the media does not drain too fast or too slow.

25. The method of claim 20 wherein the descending streams, sheets or droplets of media free

26. The method of claim 20 wherein the descending streams, sheets or droplets of media travel downward along a string, wire, sheet or mesh.

27. The method of claim 20 wherein the media is re-circulated from the collector to the header using a pump or air-lift.

28. The method of claim 20 further comprising providing an inclined collector plate positioned above the collector.

29. The method of claim 20 further comprising providing one or more turbines above the collector.

30. The method of claim 20 further wherein steps (ii) through (v) are repeated multiple times until a target organism growth density in the media is achieved.
